(19) United States
(12) Patent Application Publication
(10) Pub. No.: US 2025/0281269 A1
(43) Pub. Date: Sep. 11, 2025

(54) CUSTOMIZED DENTAL PROSTHESIS FLEXIBLE INTELLIGENT MANUFACTURING METHOD AND SYSTEM THEREOF

(71) Applicants: BETAMARS DENTALLAB CO., LTD., Zhuhai (CN); DIGITAL DIRECT DENTAL, Zhuhai (CN)

(72) Inventors: Wenhu LIU, Zhuhai (CN); Tie BIN, Zhuhai (CN); Zhiyuan LIU, Zhuhai (CN); Qiuxia LI, Zhuhai (CN); Guoying XIAO, Zhuhai (CN)

(73) Assignees: BETAMARS DENTALLAB CO., LTD., Zhuhai (CN); DIGITAL DIRECT DENTAL, Zhuhai (CN)

(21) Appl. No.: 19/215,744

(22) Filed: May 22, 2025

Related U.S. Application Data
(63) Continuation-in-part of application No. 18/764,261, filed on Jul. 4, 2024.
Application No. 18/764,261 is a continuation of application No. PCT/CN2023/089119, filed on Apr. 19, 2023.

(30) Foreign Application Priority Data
Jun. 22, 2022 (CN) 202210708257.7

(51) Int. Cl. A61C 13/00

(52) U.S. Cl. CPC A61C 13/0004; A61C 13/0022

(57) ABSTRACT

Disclosed are a customized dental prosthesis flexible intelligent manufacturing method and a system thereof. The method includes: retrieving a 3D dental prosthesis model file onto a 3D zirconia block graphic file, performing layout arrangement, and generating a toolpath file; assigning a unique identifier to each dental prosthesis based on the layout-arranged graphic file, setting coordinates for each dental prosthesis, and generating a coordinate file; transmitting the coordinate file to a prosthesis removal device and the toolpath file to a carving device via a server, wherein the carving device grasps a corresponding zirconia block for cutting; transporting the cut zirconia block to a prosthesis removal station, where the prosthesis removal device moves the zirconia block to a corresponding coordinate position according to the coordinate file to perform cutting operations on connecting rods, transfers the cut dental prosthesis to a sintering device, and uploads the coordinate file to the server.

### CROSS-REFERENCE TO RELATED APPLICATIONS

The present application is a continuation-in-part of U.S. patent application Ser. No. 18/764,261, filed on Jul. 4, 2024, which is a continuation of International Patent Application No. PCT/CN2023/089119, filed on Apr. 19, 2023, which claims priority to Chinese Patent Application No. 202210708257.7, filed on Jun. 22, 2022. All of the aforementioned applications are incorporated herein by reference in their entireties.

### TECHNICAL FIELD

The present disclosure relates to the technical field of dental prosthesis processing, specifically to a customized dental prosthesis flexible intelligent manufacturing method and a system thereof.

### BACKGROUND

Dental prosthesis processing refers to the production and machining of oral prosthetic materials in the medical device industry to provide artificial oral dental restorations for patients. In existing automated dental prosthesis production lines, tracking the specific location of each tooth at individual stations is unachievable. Significant time is wasted locating corresponding teeth during production, and during calibration, teeth must be embedded into dental models for verification. However, calibration requires experienced technicians for judgment, which not only consumes substantial manpower but also inevitably leads to errors, omissions, and losses during calibration.

### SUMMARY

The present disclosure aims to overcome the shortcomings of prior art. The first objective of the present disclosure is to provide a customized dental prosthesis flexible intelligent manufacturing method that enhances production efficiency and locates each tooth's position.

The second objective of the present disclosure is to provide a customized dental prosthesis flexible intelligent manufacturing system that enhances production efficiency and locates each tooth's position.

The technical solution adopted by the present disclosure is as follows: The customized dental prosthesis flexible intelligent manufacturing method comprises following steps:
designing a 3D dental prosthesis model file and uploading the 3D dental prosthesis model file to a server;
retrieving the 3D dental prosthesis model file onto a 3D zirconia block graphic file, performing layout arrangement, and generating a toolpath file;
assigning a unique identifier to each dental prosthesis based on the layout-arranged graphic file, setting coordinates for each dental prosthesis, and generating a coordinate file;
uploading the toolpath file and the coordinate file to the server, wherein the server transmits the coordinate file to a prosthesis removal device and transmits the toolpath file to a carving device;
grasping a corresponding zirconia block by the carving device and carving the zirconia block according to the toolpath file;
transporting the carved zirconia block to a prosthesis removal station, wherein the prosthesis removal device moves the zirconia block to a corresponding coordinate position based on the coordinate file, cuts connecting rods between each dental prosthesis and the zirconia block to remove all dental prostheses from the zirconia block, delivers the dental prostheses to a sintering device, updates the coordinate file, and uploads the updated coordinate file to the server; and
sintering the dental prostheses by the sintering device.

Further, the step of assigning the unique identifier to each dental prosthesis based on the layout-arranged graphic file, setting coordinates for each dental prosthesis, and generating the coordinate file comprises:
assigning numerical identifiers to each dental prosthesis based on the layout-arranged graphic file;
defining a center of the zirconia block as a coordinate origin via a processing system, and designating a geometric center of each dental prosthesis as a positioning coordinate; and
generating the coordinate file based on the positioning coordinates.

Further, the step of grasping the corresponding zirconia block by the carving device and carving the zirconia block according to the toolpath file comprises:
sending an instruction from the server to a first robotic arm of the carving device, wherein the first robotic arm grasps the corresponding zirconia block and transfers the zirconia block to the carving device;
carving the zirconia block by the carving device according to the toolpath file; and
grasping the carved zirconia block by the first robotic arm and transferring the carved zirconia block to a conveyor line after carving is completed.

Further, the step of transporting the carved zirconia block to the prosthesis removal station, wherein the prosthesis removal device moves the zirconia block to the corresponding coordinate position based on the coordinate file, cuts connecting rods between each dental prosthesis and the zirconia block to remove all dental prostheses from the zirconia block, delivers the dental prostheses to the sintering device, updates the coordinate file, and uploads the updated coordinate file to the server comprises:
transporting the carved zirconia block via the conveyor line, wherein a second robotic arm of the prosthesis removal device grasps the zirconia block from the conveyor line and places the zirconia block at the prosthesis removal station;
moving the zirconia block to a coordinate position corresponding to a dental prosthesis to be removed based on the coordinates determined by the prosthesis removal device from the coordinate file, indicating the dental prosthesis to be removed via a laser, and cutting the connecting rod of the dental prosthesis to be removed by a removal operator;
placing the removed dental prosthesis at a designated position on a sintering tray by the removal operator after cutting the connecting rod of the dental prosthesis to be removed; and
uploading the position of the dental prosthesis on the sintering tray to the server by the prosthesis removal device to update the coordinate file of the dental prosthesis.

Further, during the step of retrieving the 3D dental prosthesis model file onto the 3D zirconia block graphic file, performing layout arrangement, and generating the toolpath file, the zirconia block is information-bound to the 3D zirconia block graphic file via a QR code.

The present disclosure also provides a customized dental prosthesis flexible intelligent manufacturing system, comprising: a server, a processing system, a carving device, a prosthesis removal device and a sintering device;

wherein the processing system, the carving device, the prosthesis removal device and the sintering device are configured to receive and send instructions;

wherein the processing system, the carving device, the prosthesis removal device and the sintering device are connected to the server;

wherein the processing system is configured to process the 3D dental prosthesis model file and the 3D zirconia block graphic file and generate files;

wherein the server is configured to receive and transmit the files;

wherein the carving device is configured to receive the toolpath file and carve the zirconia block according to the toolpath file;

wherein the prosthesis removal device is configured to receive the coordinate file, transmit the coordinate file, and cut the connecting rods of the dental prostheses based on the coordinate file; and

wherein the sintering device is configured to sinter the dental prostheses.

Further, the customized dental prosthesis flexible intelligent manufacturing system further comprises a glazing-sintering device, a sandblasting device, a polishing device and a quality inspection and packaging device;

wherein the glazing-sintering device, the sandblasting device, the polishing device and the quality inspection and packaging device are configured to receive and send instructions;

wherein the glazing-sintering device, the sandblasting device, the polishing device and the quality inspection and packaging device are connected to the server;

wherein the glazing-sintering device is configured to glaze and sinter the dental prostheses;

wherein the sandblasting device is configured to sandblast the dental prostheses;

wherein the polishing device is configured to polish the dental prostheses; and

wherein the quality inspection and packaging device is configured to inspect and package the dental prostheses.

Further, the processing system comprises a computer installed with a CAD design software, and the CAD design software is configured to upload the designed 3D dental prosthesis model file to a designated folder on the server.

Further, the carving device comprises a first robotic arm, a dental prosthesis engraving machine and a conveyor belt;

wherein the dental prosthesis engraving machine is configured to carve the dental prostheses in the zirconia block according to the toolpath file;

wherein the first robotic arm is configured to grasp the corresponding zirconia block and transfer the zirconia block to the dental prosthesis engraving machine based on instructions from the server, and further configured to place the carved zirconia block onto the conveyor belt; and

wherein the conveyor belt is configured to transport the carved zirconia block to the prosthesis removal device.

Further, the prosthesis removal device comprises a second robotic arm, a prosthesis removal station, an operation hole and a cutting device;

wherein the second robotic arm is configured to grasp the zirconia block from the conveyor belt and transport the zirconia block to the prosthesis removal station;

wherein the prosthesis removal station is configured to move the zirconia block to a corresponding coordinate position based on coordinates of each dental prosthesis, aligning a target dental prosthesis on the zirconia block with the operation hole, wherein a diameter of the operation hole is larger than a diameter of the zirconia block; and

wherein the cutting device is configured to cut the connecting rods of the dental prostheses.

Further, the prosthesis removal device further comprises a laser indicator configured to project a laser onto the dental prosthesis to be removed.

Further, an outer side of the prosthesis removal station is encased by a plurality of sealing plates, all the sealing plates enclose the prosthesis removal station to form a sealed space, and each of the sealing plates is provided with the operation hole;

the prosthesis removal device is provided with a transmission mechanism, wherein the transmission mechanism is configured to receive the zirconia block transferred by the second robotic arm and move the zirconia block to the prosthesis removal station; and

the transmission mechanism and the prosthesis removal station are partitioned by the sealing plates, each of the sealing plates is provided with a through-hole allowing passage of the transmission mechanism, and the prosthesis removal device further comprises a sealing mechanism, wherein the sealing mechanism is configured to open or seal the through-hole.

The beneficial effects of the present disclosure are as follows.

Compared to the shortcomings of prior art, in the present disclosure, operators are not permitted to manually retrieve any dental prostheses; all operations are assigned and completed by a processing system. This ensures confirmation of each dental prosthesis's specific location at every station, with the processing system tracking and maintaining relevant information for each dental prosthesis at every station throughout the entire production process. Consequently, production efficiency is improved, the phenomenon of continuously searching for dental prostheses during production is reduced, and errors, omissions, and losses during retrieval are avoided. Thus, the customized dental prosthesis flexible intelligent manufacturing method achieves enhanced production efficiency and positioning of each dental prosthesis.

The present disclosure uses a laser to indicate to a removal operator the dental prostheses requiring removal and utilizes operation holes for extraction. The operation holes are sufficiently large to facilitate easy extraction of teeth.

According to the present disclosure, the transmission mechanism is separated from the prosthesis removal station, enabling sealed treatment of the prosthesis removal station. The transmission mechanism is arranged to extend into the prosthesis removal station via the through-holes, facilitating prosthesis removal operations, thus enabling effective sealing of the prosthesis removal device and improving dust collection.

### BRIEF DESCRIPTION OF THE DRAWINGS

FIG. 1 is a schematic flowchart of a customized dental prosthesis flexible intelligent manufacturing method according to an embodiment of the present disclosure.

FIG. 2 is a schematic flowchart of a customized dental prosthesis flexible intelligent manufacturing method according to an embodiment of the present disclosure.

FIG. 3 is a schematic flowchart of a customized dental prosthesis flexible intelligent manufacturing method according to an embodiment of the present disclosure.

FIG. 4 is a schematic flowchart of a customized dental prosthesis flexible intelligent manufacturing method according to an embodiment of the present disclosure.

FIG. 5 is a structural schematic diagram of a customized dental prosthesis flexible intelligent manufacturing system according to an embodiment of the present disclosure.

FIG. 6 is a structural schematic diagram of a customized dental prosthesis flexible intelligent manufacturing system according to an embodiment of the present disclosure.

FIG. 7 is a structural schematic diagram of the connection structure of the customized dental prosthesis flexible intelligent manufacturing system from another angle according to an embodiment of the present disclosure.

FIG. 8 is a structural schematic diagram of a prosthesis removal device in the customized dental prosthesis flexible intelligent manufacturing system according to an embodiment of the present disclosure.

FIG. 9 is a partial exploded view of the prosthesis removal device in the customized dental prosthesis flexible intelligent manufacturing system according to an embodiment of the present disclosure.

### DETAILED DESCRIPTION OF THE EMBODIMENTS

Embodiments of the present disclosure will be described in more detail below with reference to the accompanying drawings. Although certain embodiments of the present disclosure are shown in the drawings, it should be understood that the present disclosure may be implemented in various forms and should not be construed as limited to the embodiments set forth herein. These embodiments are provided to ensure a more thorough and complete understanding of the present disclosure. It should be understood that the drawings and embodiments of the present disclosure are for illustrative purposes only and are not intended to limit the scope of protection of the present disclosure.

It should be understood that the steps described in the method embodiments of the present disclosure may be performed in different orders and/or in parallel. Additionally, method embodiments may include additional steps and/or omit performing illustrated steps. The scope of the present disclosure is not limited in this respect.

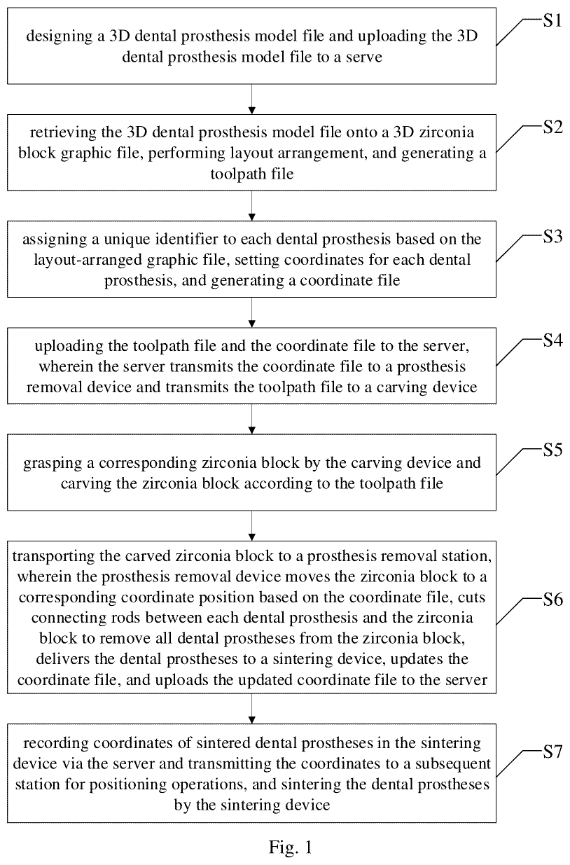

As shown in FIG. 1, the present disclosure provides a customized dental prosthesis flexible intelligent manufacturing method. The method comprises following steps.

Step S1, designing a three-dimensionality (3D) dental prosthesis model file and uploading the 3D dental prosthesis model file to a server.

Specifically, the 3D dental prosthesis model file is designed based on a computer-aided design (CAD) software and uploaded to a designated folder on the server.

Step S2, retrieving the 3D dental prosthesis model file onto a 3D zirconia block graphic file, performing layout arrangement, and generating a toolpath file.

Specifically, the 3D zirconia block graphic file is retrieved from the processing system 2, and the 3D dental prosthesis model file is retrieved from the server 1. The 3D dental prosthesis model file is retrieved onto the 3D zirconia block graphic file for layout arrangement, and a toolpath file is generated by the processing system 2. It should be noted that a layout operator retrieves the 3D zirconia block graphic file from the processing system 2. The zirconia block is provided with a QR code, and the zirconia block is information-bound to the 3D zirconia block graphic file via the QR code. The 3D zirconia block graphic file may be an empty 3D zirconia block graphic file or a 3D zirconia block graphic file containing partial dental prostheses. During the step of the 3D dental prosthesis model file is retrieved onto the 3D zirconia block graphic file for layout arrangement, one or a plurality of 3D dental prosthesis model files are retrieved onto the 3D zirconia block graphic file via the processing system 2 for layout arrangement until the 3D zirconia block graphic file is fully loaded with the 3D dental prosthesis model file.

Step S3, assigning a unique identifier to each dental prosthesis based on the layout-arranged graphic file, setting coordinates for each dental prosthesis, and generating a coordinate file.

Specifically, numerical identifiers are assigned to each dental prosthesis. For example, if the zirconia block contains 10 dental prostheses, the 10 dental prostheses are numbered (1, 2, 3, 4, 5, 6, 7, 8, 9, 10). With the center of the zirconia block defined as the origin, the geometric center of each dental prosthesis is selected via the processing system 2 to set positioning coordinates, and a coordinate file is generated.

Step S4, uploading the toolpath file and the coordinate file to the server, wherein the server transmits the coordinate file to a prosthesis removal device and transmits the toolpath file to a carving device.

Specifically, after the coordinate file is finalized and uploaded to the server, the coordinates of all dental prostheses on the zirconia block are acquired by the server. Upon transmission of the toolpath file to the carving device by the server, carving operations are performed on the dental prostheses within the zirconia block by the carving device according to the toolpath file.

Step S5, grasping a corresponding zirconia block by the carving device and carving the zirconia block according to the toolpath file.

Step S6, transporting the carved zirconia block to a prosthesis removal station, wherein the prosthesis removal device moves the zirconia block to a corresponding coordinate position based on the coordinate file, cuts connecting rods between each dental prosthesis and the zirconia block to remove all dental prostheses from the zirconia block, delivers the dental prostheses to a sintering device, updates the coordinate file, and uploads the updated coordinate file to the server.

Specifically, a robotic arm of the prosthesis removal device grasps the zirconia block from a conveyor line and transfers the zirconia block to the prosthesis removal station. The prosthesis removal device confirms the coordinate values of each dental prosthesis via the coordinate file, moves the zirconia block to the corresponding coordinate position to align the corresponding dental prosthesis with an operation hole, and employs a laser to indicate the dental prosthesis. A removal operator cuts the connecting rod of the indicated dental prosthesis through the operation hole, extracts the dental prosthesis, and places the dental prosthesis at a designated position on a sintering tray. The removal sequence follows the numerical identifiers of the dental prostheses. During removal, the coordinates of dental prostheses on the sintering tray are updated by the prosthesis removal device, and the updated coordinate file is uploaded to the server.

Step S7, recording coordinates of sintered dental prostheses in the sintering device via the server and transmitting the coordinates to a subsequent station for positioning operations, and sintering the dental prostheses by the sintering device.

Further, subsequent operations in the glazing-sintering device, the sandblasting device, the polishing device, and the quality inspection and packaging device are performed in the aforementioned manner to track real-time coordinates of dental prostheses at each station.

It should be noted that throughout the process, operators are not permitted to manually retrieve any dental prostheses; all operations are assigned and completed by a processing system. This ensures confirmation of each dental prosthesis's specific location at every station, with the processing system tracking and maintaining relevant information for each dental prosthesis at every station throughout the entire production process. Consequently, production efficiency is improved, the phenomenon of continuously searching for dental prostheses during production is reduced, and errors, omissions, and losses during retrieval are avoided. Thus, the customized dental prosthesis flexible intelligent manufacturing method achieves enhanced production efficiency and positioning of each dental prosthesis.

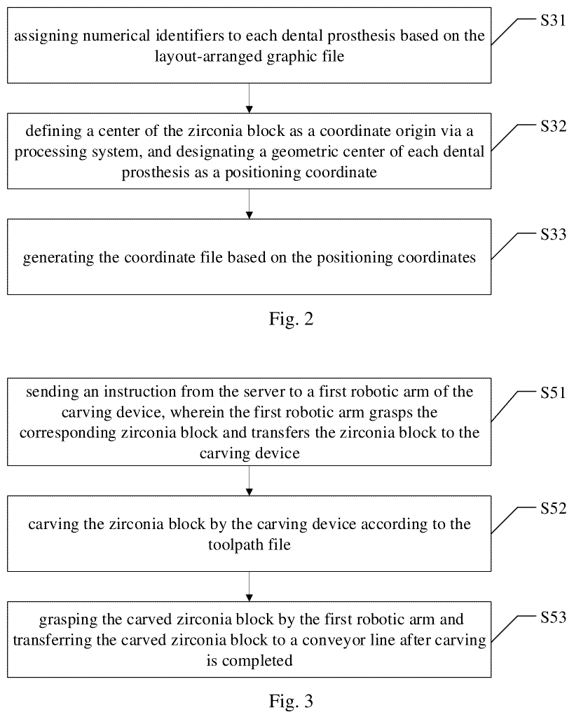

In the above Step S3, as shown in FIG. 2, the step of assigning the unique identifier to each dental prosthesis based on the layout-arranged graphic file, setting coordinates for each dental prosthesis, and generating the coordinate file comprises the follows.

Step S31, assigning numerical identifiers to each dental prosthesis based on the layout-arranged graphic file.

Step S32, defining a center of the zirconia block as a coordinate origin via a processing system, and designating a geometric center of each dental prosthesis as a positioning coordinate. Step S33, generating the coordinate file based on the positioning coordinates.

In the above Step S5, as shown in FIG. 3, the step of grasping the corresponding zirconia block by the carving device and carving the zirconia block according to the toolpath file comprises the follows.

Step S51, sending an instruction from the server to a first robotic arm of the carving device, wherein the first robotic arm grasps the corresponding zirconia block and transfers the zirconia block to the carving device.

Step S52, carving the zirconia block by the carving device according to the toolpath file.

Step S53, grasping the carved zirconia block by the first robotic arm and transferring the carved zirconia block to a conveyor line after carving is completed.

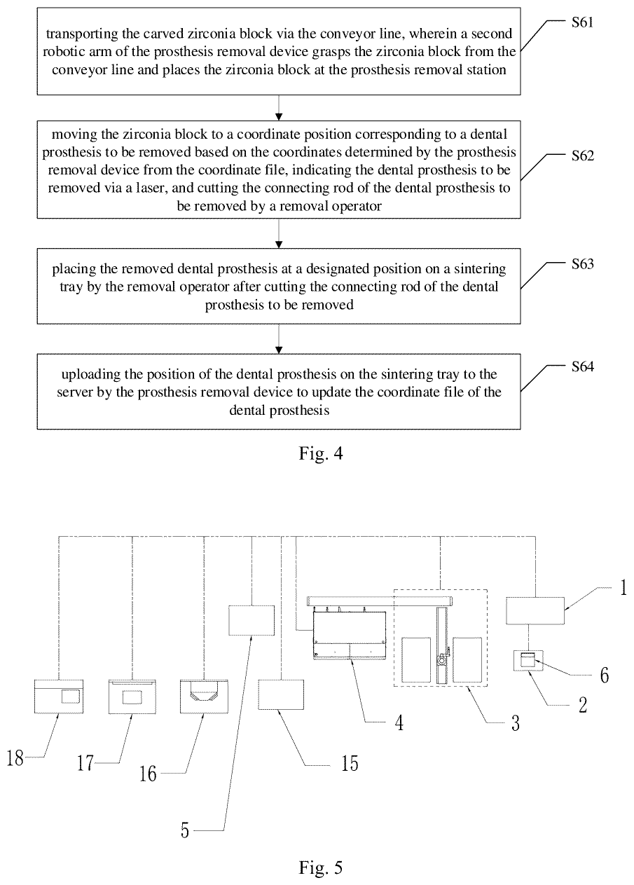

In the above Step S6, as shown in FIG. 4, the step of transporting the carved zirconia block to the prosthesis removal station, wherein the prosthesis removal device moves the zirconia block to the corresponding coordinate position based on the coordinate file, cuts connecting rods between each dental prosthesis and the zirconia block to remove all dental prostheses from the zirconia block, delivers the dental prostheses to the sintering device, updates the coordinate file, and uploads the updated coordinate file to the server comprises the follows:

Step S61, transporting the carved zirconia block via the conveyor line, wherein a second robotic arm of the prosthesis removal device grasps the zirconia block from the conveyor line and places the zirconia block at the prosthesis removal station.

Step S62, moving the zirconia block to a coordinate position corresponding to a dental prosthesis to be removed based on the coordinates determined by the prosthesis removal device from the coordinate file, indicating the dental prosthesis to be removed via a laser, and cutting the connecting rod of the dental prosthesis to be removed by a removal operator.

Step S63, placing the removed dental prosthesis at a designated position on a sintering tray by the removal operator after cutting the connecting rod of the dental prosthesis to be removed

Step S64, uploading the position of the dental prosthesis on the sintering tray to the server by the prosthesis removal device to update the coordinate file of the dental prosthesis.

Specifically, the zirconia block is moved to the corresponding coordinate position by the prosthesis removal device based on the coordinate values of each dental prosthesis determined by the layout operator and generated by the server for the removal operator to perform operations. Thereby, the corresponding dental prosthesis of the zirconia block is aligned with the operation hole, enabling the operator to cut the connecting rod between the dental prosthesis and the zirconia block through the operation hole and extract the dental prosthesis from the operation hole.

In the above Step S2, during the step of retrieving the 3D dental prosthesis model file onto the 3D zirconia block graphic file, performing layout arrangement, and generating the toolpath file, the zirconia block is information-bound to the 3D zirconia block graphic file via a QR code.

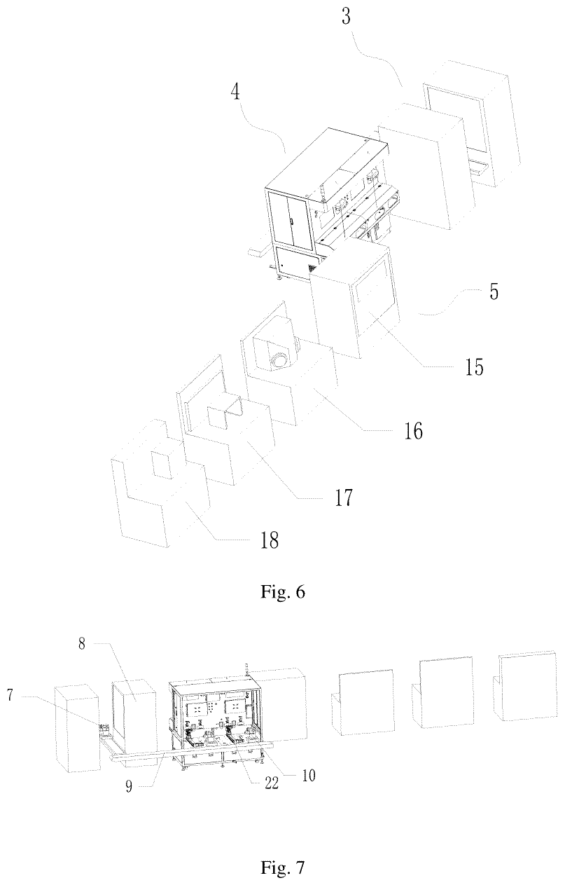

In addition, as shown in FIGS. 5, 6, and 7, the present disclosure also provides a customized dental prosthesis flexible intelligent manufacturing system, comprising: a server 1, a processing system 2, a carving device 3, a prosthesis removal device 4 and a sintering device 5;

wherein the processing system 2, the carving device 3, the prosthesis removal device 4 and the sintering device 5 are configured to receive and send instructions;

wherein the processing system 2, the carving device 3, the prosthesis removal device 4 and the sintering device 5 are connected to the server 1;

wherein the processing system 2 is configured to process the 3D dental prosthesis model file and the 3D zirconia block graphic file and generate files;

wherein the server 1 is configured to receive and transmit the files;

wherein the carving device 3 is configured to receive the toolpath file and carve the zirconia block according to the toolpath file;

wherein the prosthesis removal device 4 is configured to receive the coordinate file, transmit the coordinate file, and cut the connecting rods of the dental prostheses based on the coordinate file; and wherein the sintering device 5 is configured to sinter the dental prostheses.

In some embodiments, the customized dental prosthesis flexible intelligent manufacturing system, further comprising: a glazing-sintering device 15, a sandblasting device 16, a polishing device 17 and a quality inspection and packaging device 18;

wherein the glazing-sintering device 15, the sandblasting device 16, the polishing device 17 and the quality inspection and packaging device 18 are configured to receive and send instructions;

wherein the glazing-sintering device 15, the sandblasting device 16, the polishing device 17 and the quality inspection and packaging device 18 are connected to the server 1;

wherein the glazing-sintering device 15 is configured to glaze and sinter the dental prostheses;

wherein the sandblasting device 16 is configured to sandblast the dental prostheses;

wherein the polishing device 17 is configured to polish the dental prostheses; and

wherein the quality inspection and packaging device 18 is configured to inspect and package the dental prostheses.

During use, operators design the 3D dental prosthesis model file via the processing system 2 and upload the 3D dental prosthesis model file to a designated folder on the server 1. A layout operator retrieves the 3D zirconia block graphic file from the processing system 2 and retrieves the 3D dental prosthesis model file from the server 1, then retrieves the 3D dental prosthesis model file onto the 3D zirconia block graphic file for layout arrangement. A toolpath file is generated by the processing system 2. Numerical identifiers are assigned to each dental prosthesis, coordinates are set for each dental prosthesis, and a coordinate file is generated based on the layout-arranged graphic file. The toolpath file and the coordinate file are uploaded to the server 1. The server 1 transmits the coordinate file to the prosthesis removal device 4 and the toolpath file to the carving device 3. The carving device 3 grasps the corresponding zirconia block for carving. The carved zirconia block is transported to the prosthesis removal station 11. The prosthesis removal device 4 moves the zirconia block to the corresponding coordinate position according to the coordinate file, enabling the operator to perform the connecting rod cutting operations. The dental cut prostheses are extracted to the sintering device 5, and the coordinate file is uploaded to the server 1 by the prosthesis removal device 4. The server 1 records the coordinates of sintered dental prostheses in the sintering device and transmits the coordinates to a subsequent station for positioning operations. The sintering device sinters the dental prostheses. Subsequent operations in the glazing-sintering device, the sandblasting device, the polishing device, and the quality inspection and packaging device are performed in the aforementioned manner to track real-time coordinates of dental prostheses at each station. Therefore, the customized dental prosthesis flexible intelligent manufacturing system ensures confirmation of each dental prosthesis's specific location at every station, with the processing system 2 tracking and maintaining relevant information for each dental prosthesis at every station throughout the entire production process. Consequently, production efficiency is improved, the phenomenon of continuously searching for dental prostheses during production is reduced, and errors, omissions, and losses during retrieval are avoided. Thus, the customized dental prosthesis flexible intelligent manufacturing system achieves enhanced production efficiency and positioning of each dental prosthesis.

As shown in FIG. 5, in some embodiments, the processing system 2 comprises a computer 6 installed with a CAD design software, and the CAD design software is configured to upload the designed 3D dental prosthesis model file to a designated folder on the server 1.

As shown in FIG. 7, in some embodiments, the carving device 3 comprises a first robotic arm 7, a dental prosthesis engraving machine 8 and a conveyor belt 9; wherein the dental prosthesis engraving machine 8 to perform carving and cutting operations on the dental prostheses in the zirconia block; the first robotic arm 7 is configured to grasp the corresponding zirconia block based on instructions from the server 1, transfer the zirconia block to the dental prosthesis engraving machine 8 for carving and cutting, and place the carved zirconia block onto the conveyor belt 9; and the conveyor belt 9 is configured to transport the carved zirconia block to the prosthesis removal device 4.

As shown in FIGS. 5 to 9, in some embodiments, the prosthesis removal device 4 comprises a second robotic arm 10, a prosthesis removal station 11, a removal operation hole 12 and a cutting device 13. The second robotic arm 10 is configured to grasp the zirconia block from the conveyor belt 9 and transfer the zirconia block to the prosthesis removal station 11. The prosthesis removal station 11 is configured to move the zirconia block to a corresponding coordinate position based on coordinate values of each dental prosthesis, such that a corresponding dental prosthesis on the zirconia block is positioned below the removal operation hole 12. The cutting device 13 is configured to cut connecting rods of the corresponding dental prosthesis. An operator can extend a hand into the operation hole to perform cutting operations on the connecting rods using the cutting device 13.

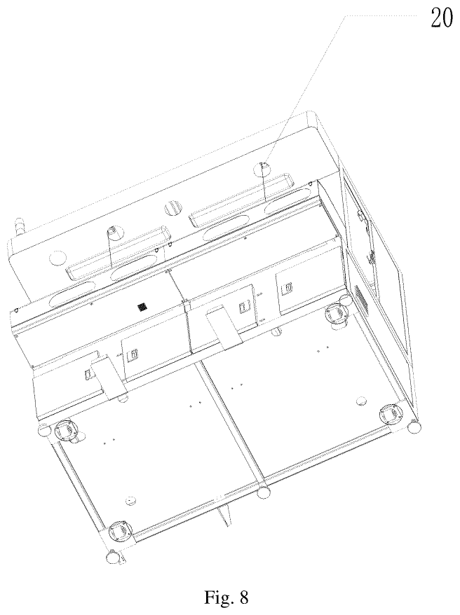

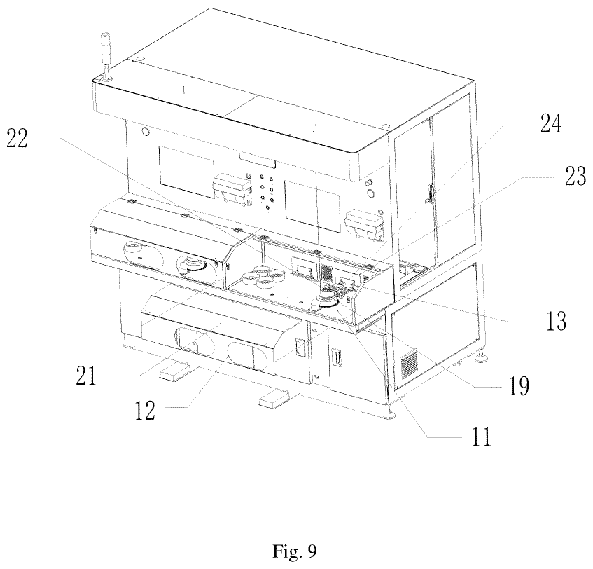

As shown in FIG. 8 and FIG. 9, in some embodiments, the prosthesis removal device 4 further comprises a laser indicator 20. The laser indicator 20 is provided on the prosthesis removal device 4 and positioned above the prosthesis removal station 11. The laser indicator 20 is configured to emit a laser beam onto the zirconia block 19 at the prosthesis removal station 11 to indicate a next dental prosthesis to be removed.

In some embodiments, an outer side of the prosthesis removal station 11 is encased by a plurality of sealing plates 21, the sealing plates 21 enclose the prosthesis removal station 11 to form a sealed space, and each of the sealing plates 21 is provided with the operation hole 12, wherein the operation hole 12 is generally located on a front side or upper side of the prosthesis removal station 11.

The prosthesis removal device 4 is further provided with a transmission mechanism 22, wherein the transmission mechanism 22 is configured to receive the zirconia block 19 transferred by the second robotic arm 10 and move the zirconia block 19 to the prosthesis removal station 11.

Since the prosthesis removal station 11 is sealed by the sealing plates 21, to allow the transmission mechanism 22 to enter the prosthesis removal station 11, a through-hole 23 is formed on a side of the sealing plates 21 facing the transmission mechanism 22. A sealing mechanism 24 is disposed at the through-hole 23, and the sealing mechanism 24 is configured to open or seal the through-hole 23. When the through-hole 23 is open, the transmission mechanism 22 drives the zirconia block 19 into the prosthesis removal station 11.

The prosthesis removal station 11 is provided with a dust collection mechanism inside. The dust collection mechanism is configured to suction debris within the prosthesis removal station 11 generated during the dental prosthesis removal process. Through the aforementioned structure, the sealing performance of the prosthesis removal station 11 is enhanced, and the dust collection effectiveness is improved.

Currently, when cutting zirconia crowns, to prevent the crowns from falling off or shifting during the cutting process, several support rods are reserved to fix the crowns relative to the material throughout the entire cutting process. After cutting, the support rods are manually severed to detach the crowns from the material, and residual support rods on the crowns are polished. Since these operations are performed manually, manual polishing often damages the crown body, causing deviations from the designed shape and increasing labor costs.

To address this, this embodiment provides a dental prosthesis manufacturing method that facilitates dental prosthesis removal, comprising following steps.

Step S1, designing a 3D dental prosthesis model file and uploading the 3D dental prosthesis model file to a server.

Step S2, retrieving the 3D dental prosthesis model file onto a 3D zirconia block graphic file, performing layout arrangement, and generating a toolpath file.

Step S3, assigning a unique identifier to each dental prosthesis based on the layout-arranged graphic file, setting coordinates for each dental prosthesis, and generating a coordinate file.

Step S4, uploading the toolpath file and the coordinate file to the server, wherein the server transmits the coordinate file to a prosthesis removal device and transmits the toolpath file to a carving device.

Step S5, grasping a corresponding zirconia block by the carving device and carving the zirconia block according to the toolpath file.

During carving, the method includes: after cutting a reverse side (inner crown surface) of the dental prosthesis, press a flexible adhesive onto the inner crown surface, apply a magnetic field to the flexible adhesive to solidify the flexible adhesive, fill the inner crown surface, and form support points connected to device within the inner crown surface.

Subsequently, connect the dental prosthesis via the support points, flip the dental prosthesis to carve and cut a front side of the dental prosthesis. After carving the front side of the dental prosthesis, cease applying the magnetic field to soften the flexible adhesive, causing the dental prosthesis to detach. The detached dental prosthesis is caught by a soft pad to obtain a dental prosthesis conforming to the designed shape.

Step S6, sintering the dental prostheses by the sintering device.

It should be noted that this embodiment corresponds to the system embodiments associated with the aforementioned method embodiments. This embodiment can be implemented in conjunction with the aforementioned method embodiments. Technical details mentioned in the method embodiments remain valid in this embodiment and are not reiterated to avoid redundancy. Similarly, technical details mentioned in this embodiment may also be applied to the aforementioned method embodiments.

While the embodiments of the present disclosure are described with reference to specific implementations, they are not intended to limit the scope of the present disclosure. For those skilled in the art, modifications to the described embodiments and combinations with other solutions will be apparent based on this specification.

What is claimed is:

1. A customized dental prosthesis flexible intelligent manufacturing method, comprising following steps:
designing a three-dimensionality (3D) dental prosthesis model file and uploading the 3D dental prosthesis model file to a server;
retrieving the 3D dental prosthesis model file onto a 3D zirconia block graphic file, performing layout arrangement, and generating a toolpath file;
assigning a unique identifier to each dental prosthesis based on the layout-arranged graphic file, setting coordinates for each dental prosthesis, and generating a coordinate file;
uploading the toolpath file and the coordinate file to the server, wherein the server transmits the coordinate file to a prosthesis removal device and transmits the toolpath file to a carving device;
grasping a corresponding zirconia block by the carving device and carving the zirconia block according to the toolpath file;
transporting the carved zirconia block to a prosthesis removal station, wherein the prosthesis removal device moves the zirconia block to a corresponding coordinate position based on the coordinate file, cuts connecting rods between each dental prosthesis and the zirconia block to remove all dental prostheses from the zirconia block, delivers the dental prostheses to a sintering device, updates the coordinate file, and uploads the updated coordinate file to the server; and
sintering the dental prostheses by the sintering device.

2. The customized dental prosthesis flexible intelligent manufacturing method according to claim 1, wherein the step of assigning the unique identifier to each dental prosthesis based on the layout-arranged graphic file, setting coordinates for each dental prosthesis, and generating the coordinate file comprises:
assigning numerical identifiers to each dental prosthesis based on the layout-arranged graphic file:
defining a center of the zirconia block as a coordinate origin via a processing system, and designating a geometric center of each dental prosthesis as a positioning coordinate; and
generating the coordinate file based on the positioning coordinates.

3. The customized dental prosthesis flexible intelligent manufacturing method according to claim 1, wherein the step of grasping the corresponding zirconia block by the carving device and carving the zirconia block according to the toolpath file comprises:
sending an instruction from the server to a first robotic arm of the carving device, wherein the first robotic arm grasps the corresponding zirconia block and transfers the zirconia block to the carving device;
carving the zirconia block by the carving device according to the toolpath file; and
grasping the carved zirconia block by the first robotic arm and transferring the carved zirconia block to a conveyor line after carving is completed.

4. The customized dental prosthesis flexible intelligent manufacturing method according to claim 1, wherein the step of transporting the carved zirconia block to the prosthesis removal station, wherein the prosthesis removal device moves the zirconia block to the corresponding coordinate position based on the coordinate file, cuts connecting rods between each dental prosthesis and the zirconia block to remove all dental prostheses from the zirconia block, delivers the dental prostheses to the sintering device, updates the coordinate file, and uploads the updated coordinate file to the server comprises:
transporting the carved zirconia block via the conveyor line, wherein a second robotic arm of the prosthesis removal device grasps the zirconia block from the conveyor line and places the zirconia block at the prosthesis removal station;
moving the zirconia block to a coordinate position corresponding to a dental prosthesis to be removed based on the coordinates determined by the prosthesis removal device from the coordinate file, indicating the dental prosthesis to be removed via a laser, and cutting the connecting rod of the dental prosthesis to be removed by a removal operator;
placing the removed dental prosthesis at a designated position on a sintering tray by the removal operator after cutting the connecting rod of the dental prosthesis to be removed; and
uploading the position of the dental prosthesis on the sintering tray to the server by the prosthesis removal device to update the coordinate file of the dental prosthesis.

5. The customized dental prosthesis flexible intelligent manufacturing method according to claim 1, wherein during the step of retrieving the 3D dental prosthesis model file onto the 3D zirconia block graphic file, performing layout arrangement, and generating the toolpath file, the zirconia block is information-bound to the 3D zirconia block graphic file via a QR code.

6. A customized dental prosthesis flexible intelligent manufacturing system, comprising: a server, a processing system, a carving device, a prosthesis removal device and a sintering device;
wherein the processing system, the carving device, the prosthesis removal device and the sintering device are configured to receive and send instructions;
the processing system, the carving device, the prosthesis removal device and the sintering device are connected to the server;
the processing system is configured to process the 3D dental prosthesis model file and the 3D zirconia block graphic file and generate files;
the server is configured to receive and transmit the files;
the carving device is configured to receive the toolpath file and carve the zirconia block according to the toolpath file;
the prosthesis removal device is configured to receive the coordinate file, transmit the coordinate file, and cut the connecting rods of the dental prostheses based on the coordinate file; and
the sintering device is configured to sinter the dental prostheses.

7. The customized dental prosthesis flexible intelligent manufacturing system according to claim 6, further comprising: a glazing-sintering device, a sandblasting device, a polishing device and a quality inspection and packaging device;
wherein the glazing-sintering device, the sandblasting device, the polishing device and the quality inspection and packaging device are configured to receive and send instructions;
the glazing-sintering device, the sandblasting device, the polishing device and the quality inspection and packaging device are connected to the server;
the glazing-sintering device is configured to glaze and sinter the dental prostheses;
the sandblasting device is configured to sandblast the dental prostheses;
the polishing device is configured to polish the dental prostheses; and
the quality inspection and packaging device is configured to inspect and package the dental prostheses.

8. The customized dental prosthesis flexible intelligent manufacturing system according to claim 6, wherein the processing system comprises a computer installed with a computer-aided design (CAD) design software, and the CAD design software is configured to upload the designed 3D dental prosthesis model file to a designated folder on the server.

9. The customized dental prosthesis flexible intelligent manufacturing system according to claim 8, wherein the carving device comprises a first robotic arm, a dental prosthesis engraving machine and a conveyor belt;
the dental prosthesis engraving machine is configured to carve the dental prostheses in the zirconia block according to the toolpath file;
the first robotic arm is configured to grasp the corresponding zirconia block and transfer the zirconia block to the dental prosthesis engraving machine based on instructions from the server, and further configured to place the carved zirconia block onto the conveyor belt; and
the conveyor belt is configured to transport the carved zirconia block to the prosthesis removal device.

10. The customized dental prosthesis flexible intelligent manufacturing system according to claim 9, wherein the prosthesis removal device comprises a second robotic arm, a prosthesis removal station, an operation hole and a cutting device;
the second robotic arm is configured to grasp the zirconia block from the conveyor belt and transport the zirconia block to the prosthesis removal station;
the prosthesis removal station is configured to move the zirconia block to a corresponding coordinate position based on coordinates of each dental prosthesis, aligning a target dental prosthesis on the zirconia block with the operation hole, a diameter of the operation hole is larger than a diameter of the zirconia block; and
the cutting device is configured to cut the connecting rods of the dental prostheses.

11. The customized dental prosthesis flexible intelligent manufacturing system according to claim 10, wherein the prosthesis removal device further comprises a laser indicator configured to project a laser onto the dental prosthesis to be removed.

12. The customized dental prosthesis flexible intelligent manufacturing system according to claim 10, wherein an outer side of the prosthesis removal station is encased by a plurality of sealing plates, all the sealing plates enclose the prosthesis removal station to form a sealed space, and each of the sealing plates is provided with the operation hole;
the prosthesis removal device is provided with a transmission mechanism, the transmission mechanism is configured to receive the zirconia block transferred by the second robotic arm and move the zirconia block to the prosthesis removal station; and
the transmission mechanism and the prosthesis removal station are partitioned by the sealing plates, each of the sealing plates is provided with a through-hole allowing passage of the transmission mechanism, and the prosthesis removal device further comprises a sealing mechanism, the sealing mechanism is configured to open or seal the through-hole.
